(19) United States
(12) Patent Application Publication
(10) Pub. No.: US 2026/0153522 A1
(43) Pub. Date: Jun. 4, 2026

(54) METHOD TO DETERMINE IMPACT ON ANALYTES DUE TO CYTOLYSIS IN A SAMPLE

(71) Applicant: Radiometer Medical ApS, Brønshøj (DK)

(72) Inventors: Michael Taagaard, Brønshøj (DK); Peter Frischauf, Brønshøj (DK)

(21) Appl. No.: 18/714,916

(22) PCT Filed: Dec. 2, 2022
(86) PCT No.: PCT/EP2022/084251

§ 371 Date: May 30, 2024

(30) Foreign Application Priority Data
Dec. 3, 2021 (EP) 21212324.4

(51) Int. Cl. G01N 33/72; G01N 33/50; G01N 33/84; G01N 33/96

(52) U.S. Cl. CPC G01N 33/726; G01N 33/5005; G01N 33/84; G01N 33/96; G01N 2333/805

(57) ABSTRACT

Disclosed is a method and device/apparatus for determination of the contribution from lysed cells to measurement values of an analyte present in the extracellular compartment. The method employs multiple tandem measurements of analyte and an intracellular marker from samples where different numbers of cells are lysed so as to establish the relationship between the measured value of the intracellular marker and the analyte.

### FIELD OF THE INVENTION

The present invention relates to the field of sample testing. More particularly, the present invention relates to the field of testing of analytes in the extracellular phase in cell-containing samples such as blood samples.

### BACKGROUND OF THE INVENTION

Certain analytes of interest (e.g. electrolytes and ions) in blood samples are present in significantly different concentrations in the extracellular and intracellular compartments in vivo. For instance potassium (K+) has a normal extracellular concentration of between 3.6 and 5.2 mM the extracellular compartment in blood. In contrast, the concentration of potassium inside the red blood cell in vivo is typically between 80 and 120 mM. Sodium, on the other hand, has a normal extracellular concentration of between 135 and 145 mM, whereas the normal sodium concentration inside red blood cells is about 12 mM. These differences are maintained by active transport via dedicated channels in the cell membranes or by the fact that analytes are secreted by cells to the extracellular phase and are incapable of traversing cell membranes.

As most measurements of analytes in blood focus on the extracellular concentration, cytolysis, and in particular haemolysis, can potentially have a drastic effect on measurement results-if, for instance, handing of a blood sample causes significant haemolysis in the sample, after it has been obtained, a subsequent measurement of the analyte can either overestimate the true extracellular concentration (if the analyte is more abundant in the intracellular compartment and the cytolysis liberates the analyte to increase its apparent concentration), or underestimate the true extracellular concentration (if the analyte is less abundant in the intracellular compartment and the cytolysis dilutes the extracellular concentration).

In Koseoglu M. et al., Biochemia Medica 2011; 21(1): 79-85, it is reported that measurements of lactate dehydrogenase (LD) and aspartate aminotransferase (AST) are interfered with at almost undetectable haemolysis when using simple visual inspection (plasma haemoglobin <0.5 g/L). Clinically meaningful variations of potassium and total bilirubin were observed in moderately haemolyzed samples (haemoglobin >1 g/L). Alanine aminotransferase (ALT), cholesterol, γ-glutamyltransferase (GGT), and inorganic phosphate (P) concentrations were not interfered up to severely haemolyzed levels (haemoglobin: 2.5-4.5 g/L). Albumin, alkaline phosphatase (ALP), amylase, chloride, HDL-cholesterol, creatine kinase (CK), glucose, magnesium, total protein, triglycerides, unsaturated iron binding capacity (UIBC) and uric acid differences were statistically significant, but remained within the CLIA (Clinical Laboratory Improvement Amendments) limits: Clinically significant differences are defined as greater than total allowable error (TAE) for an analyte established by CLIA of 1988. TAE was as follows: K⁺, ±0.5 mmol/L; AST, 20%; Mg, 25%; and LDH, 20%. Percentage of results within and outside of the respective analyte reference interval (RI) was determined at each HI level. RIs were as follows: K⁺, 3.5 to 5.3 mM; AST, 10 to 37 U/l; Mg, 1.60 to 2.40 mg/dl; and LDH, 84 to 246 U/L (cf. Validation of Haemolysis Index Thresholds Optimizes Detection of Clinically Significant Haemolysis, Tanu Goyal, MD,1 and Christine L. Schmotzer, MD1,2, Am J Clin Pathol April 2015; 143:579-583).

At present, there is no international standard for handling the contribution to measured results from haemolysis in a sample, and most hospitals simply use the thresholds set by the manufacturers of the chemical analyzers to determine whether a sample contains too high or too low concentrations of analyte. All blood samples for analysis of haemolysis in the sample are today send to central labs where the analysis is performed at plasma samples. Regarding the POC-areas (point-of-care-areas) and analysis of haemolysis in a whole blood sample there is no commercial product on the market.

The above describe the current situation for detection and identifying haemolysis in samples. There is however a need for providing information to the user regarding the impact of haemolysis in the sample, in order to provide an indication of the true value of measured parameters.

It has been attempted to correct e.g. potassium concentrations measured in samples: “Correction Factors for Estimating Potassium Concentrations in Samples With In Vitro Haemolysis”, Mai M. H. Mansour, MS; Hassan M. E. Azzazy, PhD, DABCC; Steven C. Kazmierczak, PhD, DABCC, Arch Pathol Lab Med—Vol 133, June 2009. This article from 2009 describes very accurately the situation and the hurdles regarding a correction factor for potassium. The authors identified 744 articles for considerations and 43 for systematic review. The conclusion from the article is that use of correction factors for estimating the true potassium concentration in samples with evidence of in vitro haemolysis is not recommended and this reflects even the current situation where potassium values are not corrected due to haemolysis in the sample.

WO 2018/121459 discloses a method for determining in vitro haemolysis and correction of at least one blood parameter in a blood sample by correcting for the in vitro haemolysis. The technology disclosed in WO 2019/121459 does not provide for a determination or correction of the contribution by total cell lysis to the measured value of an analyte such as a blood parameter.

### OBJECT OF THE INVENTION

It is an object of embodiments of the invention to provide methods, a device and a computer program product for estimating true extracellular concentrations of analytes in samples and for estimating the contribution from cytolysis, in particular from haemolysis, to values measured in extracellular samples

### SUMMARY OF THE INVENTION

It has been found by the present inventors that introduction of measurements of an intracellular marker can be utilised to correct measured values of extracellular analytes. As such, it becomes possible to obtain, in situ, a corrected measurement of an analyte, which takes into consideration the impact from e.g. haemolysis on the measured value; in other words, the method of the invention provides for an in situ reference method, which can provide the true extracellular concentration (or at least a more accurate estimation thereof) as well as a direct indication of the contribution from haemolysis to the measure analyte value.

In practice, the present inventors have demonstrated that at least measurements of extracellular potassium can be corrected with high accuracy by subjecting the same sample to a series of tandem measurements of extracellular potassium and extracellular haemoglobin (also termed cell-free haemoglobin or ccfHb). By deliberately lysing a fraction of the cells of the sample in the interval between the tandem measurements, pairs of measurement values for potassium and ccfHb are obtained, whereby, e.g., a simple linear regression and subsequent extrapolation can be made. In turn, this regression model provides an estimate of the contribution to the potassium measurement value per unit of measured ccfHb (in some embodiments as a slope coefficient). In turn, in any subsequent measurement, the contribution by cell lysis to any measured value may be determined, e.g., as the product between the measured ccfHb concentration and the slope coefficient, and, e.g., by simple subtraction, also the true extracellular value can be derived.

So, in a first aspect the present invention relates to a method for determination of the impact from cell lysis on the measured concentration of an analyte present in a blood cell-containing liquid sample, the method comprising,
A) measuring the concentration of the analyte in the extracellular phase from a sample containing blood cells and measuring the concentration of an intracellular marker in the extracellular phase of the sample,
B) subsequently lysing a fraction of the blood cells followed by measuring a concentration of the analyte and a concentration of the intracellular marker in the extracellular phase of the sample containing the lysed cells,
C) optionally repeating step B one or more times, and
D) estimating the mathematical relationship between changes in the analyte concentration and the concentration of the intracellular marker at least on the basis of the pairs of measurements obtained in steps A-C, whereby the impact by cell lysis on measured concentrations of the analyte in samples is determined by calculating the part of the analyte concentration derived from cell lysis from a measured concentration of the intracellular marker.

In a related 2^{nd} aspect, the present invention relates to a method of calibration of a device for determination of extracellular analyte concentration, the method comprising carrying out, with the device, the method according to the first aspect of the invention and any embodiments thereof disclosed herein, storing in a memory module in the device a mathematical expression of relationship between changes in the analyte concentration and the concentration of the intracellular marker defined in respect of the 1^{st} aspect of the invention, so as to allow output based on subsequent measurements of analyte performed with the device to be adjusted on the basis of measured values of the intracellular marker.

A 3^{rd} aspect of the invention relates to a method of determination of analyte concentration distribution in the extracellular phase of a sample, the method comprising measuring the total concentration of the analyte in the extracellular phase and measuring the concentration of an intracellular marker in the extracellular phase, and subsequently estimating the contribution from lysis of cells to the total concentration of the analyte and/or correcting the measured analyte concentration value on the basis of the measured concentration of the intracellular marker, wherein the mathematical relationship between analyte concentration and concentration of the intracellular marker has been established according to the method of the first or second aspect of the invention as well as any embodiments thereof disclosed herein.

A 4^{th} aspect of the invention relates to a device for determination of analyte concentration distribution in the extracellular phase of a sample, wherein the device is configured to measure the total concentration of the analyte in the extracellular phase and measure the concentration of an intracellular marker in the extracellular phase, and subsequently estimating, such as calculating, the contribution from lysis of cells to the total concentration of the analyte by inter- or extrapolation from the measured concentration of the intracellular marker based on a mathematical or functional relationship between analyte concentration and concentration of the intracellular marker.

A 5^{th} aspect of the invention relates to a computer program or computer program product comprising instructions to cause the device of the 4^{th} aspect to measure the total concentration of the analyte in the extracellular phase of a sample and to measure the concentration of an intracellular marker in the extracellular phase of the sample, and subsequently to estimate, such as calculate, the contribution from lysis of cells to the total concentration of the analyte by inter- or extrapolation from the measured concentration of the intracellular marker based on a mathematical or functional relationship between analyte concentration and concentration of the intracellular marker.

A 6^{th} aspect of the invention relates to a computer implemented method comprising the steps of:
a) Obtaining first concentrations in the form of a first total concentration of an analyte in an extracellular phase of a sample and a first concentration of an intracellular marker in the extracellular phase of the sample;
b) Obtaining second concentrations in the form of a second concentration of the analyte and a second concentration of the intracellular marker in the extracellular phase of the same sample, wherein a fraction of the cells in the sample has been lysed prior to obtaining the second concentrations;
c) estimating the relationship between changes in the analyte concentration and the concentration of the intracellular marker on the basis of at least the first and second concentrations obtained in steps a) and b).

In an alternative formulation of a 6^{th} aspect there is presented a computer implemented method for determination of the impact from cell lysis on the measured concentration of an analyte present in a blood cell-containing liquid sample, the method comprising,
A) obtaining the concentration of the analyte in the extracellular phase from a sample containing blood cells and measuring the concentration of an intracellular marker in the extracellular phase of the sample,
B) subsequent to lysing a fraction of the blood cells, obtaining a concentration of the analyte and a concentration of the intracellular marker in the extracellular phase of the sample containing the lysed cells,
C) optionally repeating step B one or more times, and
D) estimating the mathematical or functional relationship between changes in the analyte concentration and the concentration of the intracellular marker at least on the basis of the pairs of measurements obtained in steps A-C, whereby the impact by cell lysis on measured concentrations of the analyte in samples is determined by estimating, such as calculating, the part of the analyte concentration derived from cell lysis from a measured concentration of the intracellular marker.

### LEGENDS TO THE FIGURE

FIG. 1 shows a graph where measured values of K⁺ and ccfHb are correlated.

FIG. 2 shows a diagram of an electrical circuit for potentiometric measurement of ions/electrolytes.

FIG. 3 shows in schematic form the principles behind a ccfHb measurement method and device.

FIG. 4 shows a schematic illustration of an exemplary device for determination of analyte concentration distribution in the extracellular phase of a (liquid) sample.

FIG. 5 shows part of an embodiment of an apparatus similar to the device of FIG. 4, but where the apparatus depicted in FIG. 5 furthermore comprises a lysing unit arranged for lysing a fraction of the blood cell.

FIG. 6 shows another embodiment of an apparatus similar to the device of FIG. 4, but where the apparatus depicted in FIG. 6 furthermore comprises a lysing unit arranged for lysing a fraction of the blood cells while being in the chamber(s) of the sensors.

### DETAILED DISCLOSURE OF THE INVENTION

### Definitions

In the present context, an “analyte” is any chemical substance, composition, or moiety (or the activity of such a chemical substance, composition, or moiety) it is of interest to determine in a sample. The typical analytes of interest are those that can or will be influenced by degree of cytolysis in a sample (see the background above), because there exists a significant difference in intracellular and extracellular concentrations of the analyte. A non-exhaustive list of analytes is provided in Table A infra. So, typical analytes are electrolytes and their related ions, extracellular enzymes and other biomolecules.

As used herein, an “intracellular marker” is a substance, which in vivo is present in negligible amounts or substantially constant amounts in the extracellular phase in a healthy individual and appears in a different and higher concentration in the intracellular phase. One example is haemoglobin, which is normally confined to the cytoplasm of red blood cells and only appears in the extracellular phase in significant amounts if the plasma membrane of red blood cells is disrupted, i.e. when there is a degree of haemolysis (that is, lysis of red blood cells). However, in principle any substance, which is confined to the cytoplasm in the living and healthy cell can act as an intracellular marker as long as it is possible to determine the extracellular concentration thereof with the necessary fidelity. The major advantage of employing haemoglobin as an intracellular marker is its abundance in whole blood, where red blood cells constitute an average of 43% v/v in healthy individuals.

In analogy, an “extracellular marker” is a substance, which in vivo is present in negligible amounts or substantially constant amounts in the intracellular phase in a healthy individual and appears in a different and higher concentration in the extracellular phase. One example is albumin, which in its capacity of the most abundant plasma protein is normally confined to plasma/serum. However, in principle any substance, which is confined to the extracellular phase in a living and healthy individual can act as an extracellular marker as long as it is possible to determine the intracellular concentration thereof with the necessary fidelity.

The expression “impact from cell lysis” refers to the change—positive or negative (or variable)—in concentration of an analyte located in the extracellular compartment, where the change in concentration is the consequence of disruption of cells (i.e. cell lysis) whereby analyte is added to the extracellular compartment (when the analyte exists in higher concentrations intracellularly than extracellularly) or whereby the analyte is diluted (when the analyte exists in lower concentrations intracellularly than extracellularly).

### SPECIFIC EMBODIMENTS OF THE INVENTION

### 1^{st} Aspect of the Invention

This aspect relates to a method for determination of the impact from cell lysis on the measured concentration of an analyte present in a blood cell-containing liquid samples (such as a blood sample or diluted blood sample), the method comprising, A) measuring the concentration of the analyte in the extracellular phase from a sample containing blood cells and measuring the concentration of an intracellular marker in the extracellular phase of the sample, B) subsequently lysing a fraction of the blood cells followed by measuring a concentration of the analyte and a concentration of the intracellular marker in the extracellular phase of the sample containing the lysed cells, C) optionally repeating step B one or more times, and D) estimating the relationship between changes in the analyte concentration and the concentration of the intracellular marker at least on the basis of the pairs of measurements obtained in steps A-C,
whereby the impact by cell lysis on measured concentrations of the analyte in samples is determined by calculating the part of the analyte concentration derived from cell lysis from a measured concentration of the intracellular marker.

Thus, this aspect includes a method for determination of the impact from cell lysis on the measured concentration of an analyte present in a blood cell-containing liquid samples (such as a blood sample or diluted blood sample), the method comprising, A) measuring the concentration of the analyte in the extracellular phase from a sample (e.g. obtained from one individual), where the sample contains blood cells, and measuring the concentration of an intracellular marker in the extracellular phase of the sample, B) subsequently lysing a fraction of the blood cells in the same sample followed by measuring a concentration of the analyte and a concentration of the intracellular marker in the extracellular phase of the same sample, now containing the newly lysed cells, C) optionally repeating step B one or more times, and D) estimating the relationship between changes in the analyte concentration and the concentration of the intracellular marker at least on the basis of the pairs of measurements obtained in steps A-C,
whereby the impact by cell lysis on measured concentrations of the analyte in samples is determined by calculating the part of the analyte concentration derived from cell lysis from a measured concentration of the intracellular marker.

An important feature of the method of the first aspect of the invention is the application of the series of lysing events taking place in steps B and C. In the prior art method disclosed in WO 2019/121459, it is described to derive a conversion factor for converting the degree of in vitro haemolysis into corresponding units of a blood parameter. According to WO 2019/121459, this conversion factor is determined by using a series of samples with a known amount of haemolysis, e.g., by mixing a non-haemolyzed sample with a completely haemolyzed sample to introduce a known amount of, e.g., free haemoglobin in the non-haemolyzed sample. The present invention avoids this derivation step by simply correlating different degrees of determined haemolysis with different determined analyte values without any prior knowledge about the degree of haemolysis in the sample and without the need to establish a series of mixed samples from a fully haemolysed sample and a non-haemolysed sample.

It is to be noted that both intracellular and extracellular makers (see the above definition) can be used according to the present invention. For an intracellular marker, there will be an increase in concentration in the extracellular phase as a consequence of cell lysis, whereas an extracellular marker will exhibit a decrease in concentration as a consequence of cell lysis—but in both cases as correlation with measured concentrations of analyte can be established. Hence, while use of intracellular markers is preferred (due to the ease of detection of deviation from very low extracellular values) all disclosure below relating to correlation with intracellular markers can be applied mutatis mutandis to properly selected extracellular markers, but it will be understood that a measured increase in an intracellular marker concentration will translate into a measured decrease in extracellular marker concentration.

Step D can e.g. be carried out by defining in a regression model or a curve-fitting model—in the form of a function where the concentration of the analyte is the dependent variable and the concentration of the intracellular marker is the independent variable—the relationship between changes in the analyte concentration and the concentration of the intracellular marker by using the pairs of measurements obtained in steps A-C, whereby the impact by cell lysis on measured concentrations of the analyte in samples is determined by calculating the part of the analyte concentration derived from cell lysis from a measured concentration of the intracellular marker.

In practice, the method of the 1^{st} aspect can establish—in a given measurement setup or measurement apparatus—a relationship between the measured value of the intracellular marker and the analyte concentration/amount. The measurements in step A in the method of the 1^{st} aspect hence serve as a baseline, and the values will typically be subtracted from the corresponding measurements values carried out in steps B and C. Thereby each given increase (ΔI) over the baseline value of the intracellular marker value can be paired with a corresponding change (Aa) of the measured analyte value. So, while this allows for determination of the “true” extracellular value and the cytolysis impact in the sample tested in the first aspect, the most interesting outcome is the establishment of the general relationship between the measured value of the intracellular marker and the measured value of the analyte, thus allowing the implementation of the invention discussed infra in the 3^{rd} aspect of the invention, where only determination in a sample of the amounts/concentrations of analyte and intracellular marker is necessary, because the measurement value of the analyte can be made subject to correction using the results obtained from the 1^{st} aspect of the invention.

Typically, the analyte is an ion or electrolyte, such as those conventionally measured when determining concentrations of electrolytes in blood samples. As such, the analyte typically is or comprises an ion selected from K⁺, Na⁺, Ca²⁺, Mg²⁺, Cl⁻, and in particular, the analyte is K⁺.

The analyte can also be a non-electrolyte, such as lactate dehydrogenase (LD), aspartate aminotransferase (AST), bilirubin, Albumin, alkaline phosphatase (ALP), amylase, HDL-cholesterol, creatine kinase (CK), glucose, total serum or plasma protein, triglycerides, unsaturated iron binding capacity (UIBC), and uric acid.

Generally, the analyte is selected from those presented in Table A, which also sets forth the effect (positive, variable, or negative) occasioned by cell lysis (in particular haemolysis) and the underlying cause of the bias:

TABLE A

| Analyte | Bias | Cause |
|---|---|---|
| Adrenocorticotropic hormone | Negative | Proteolysis |
| Activated partial thromboplastin time | Negative | Release of thromboplastic substances |
| Antithrombin | Negative | Analytical interference |
| Aspartate aminotransferase | Positive | Cellular release |
| Alanine aminotransferase | Positive | Cellular release |
| Albumin | Negative | Dilution |
| Alkaline phosphatase | Negative | Analytical interference |
| Bilirubin (neonatal) | Variable | Analytical interference |
| Bilirubin (total) | Negative | Analytical interference |
| Calcium | Negative | Dilution, protein binding |
| Calcitonine | Positive | Proteolysis |
| Chloride | Negative | Dilution |
| Cortisol | Negative | Analytical interference |
| Creatine kinase | Positive | Analytical interference |
| Creatinine | Positive | Analytical interference |
| D-dimer | Positive | Release of thromboplastic substances |
| Fibrinogen | Negative | Release of thromboplastic substances |
| Folate | Positive | Cellular release |
| Y-Glutamyltransferase | Negative | Analytical interference |
| Gastrin | Negative | Proteolysis |
| Glucagon | Negative | Proteolysis |
| Glucose | Negative | Dilution |
| Haptoglobin | Negative | Analytical interference |
| Homocysteine | Negative | Analytical interference |
| Insulin | Negative | Proteolysis |
| Iron | Positive | Analytical interference |
| Lactate dehydrogenase | Positive | Cellular release |
| Lipase | Positive | Analytical interference |
| Magnesium | Positive | Cellular release |
| Parathyroid hormone | Negative | Proteolysis |
| Phosphorus | Positive | Cellular release |
| Potassium | Positive | Cellular release |
| Prostate specific antigen | Positive | Analytical interference |
| Prothrombin time | Positive | Release of thromboplastic substances |
| Partial O₂ pressure | | |
| Partial CO₂ pressure | | |
| Sodium | Negative | Dilution |
| Urea | Positive | Cellular release |
| Testosterone | Negative | Analytical interference |
| Troponin I | Positive | Analytical interference |
| Troponin T | Negative | Analytical interference |
| Vitamin B12 | Negative | Analytical interference |

While the method of the 1^{st} aspect can be carried out on any cell containing sample, where it is of value to determine the extracellular concentration of an analyte, the preferred extracellular phase is blood serum or blood plasma, either from a non-human animal or from a human being; both of these extracellular phases may, when carrying out the method, be diluted in order to facilitate the measurement; in that case, the measurement has to be adjusted for the degree of dilution.

When the sample tested is blood, and the extracellular phase is blood serum or blood plasma, the method of the invention will provide an indication of haemolysis—other cells in blood are present in so low concentrations compared to the red blood cell concentration that the contribution from lysis of these cells (i.e. primarily white blood cells) is negligible.

As will be understood, the preferred intracellular marker is haemoglobin or a haemoglobin derivative, in particular cell-free haemoglobin (ccfHb).

The mathematical relationship estimated, e.g. the function established via the regression or curve fitting model mentioned above, is in some embodiments a linear function f(I)=a×I+b, where f(I) is the measured analyte concentration, I is the measured concentration of the intracellular marker, b is the extracellular concentration of the analyte in the absence of cell lysis, and a is the variation in extracellular concentration of the analyte per concentration unit of intracellular marker (the slope coefficient). When employing this simple model, which assumes that there exists a simple linear relationship between release of haemoglobin from lysed cells and the change in analyte concentration, the contribution from cell lysis is calculated as a×I. In other words, in this simple model, the only constant of relevance for determining the impact from cell lysis and the “true” extracellular concentration of analyte is the slope coefficient a. Importantly, since the variation in haemoglobin (Hb) content in individual red blood cells is very limited, an initial determination of the slope coefficient a allows for multiple subsequent measurements of the analyte, where the contribution from cytolysis can be readily determined.

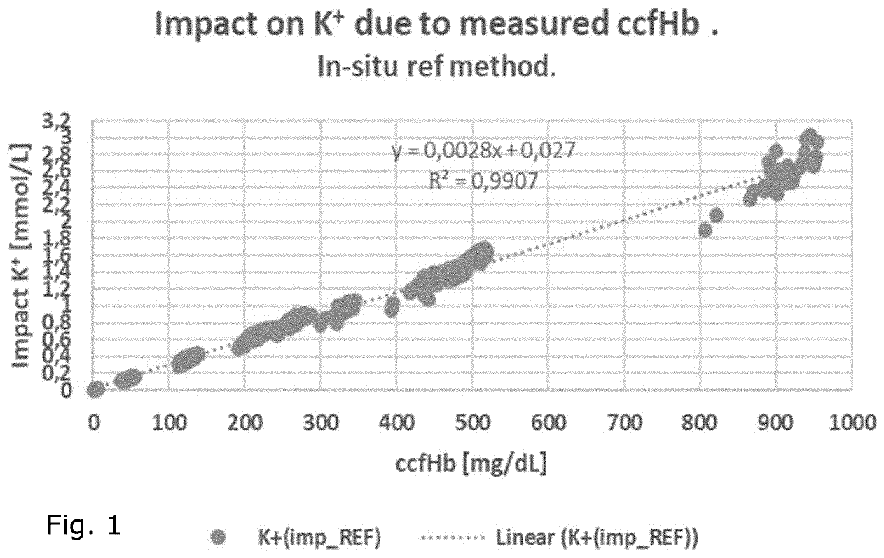

In the case of a linear relationship, the number of measurements in steps B and C can in principle be as few as 1 when assuming that a datapoint (0,0) exists (i.e. zero intracellular marker and zero analyte). In that case, the relationship derivable will be a×I and b is set to zero—this simple function thus describes the contribution from cell lysis only. However, it is preferred that a plurality of measurements is carried out to derive the a×I of a×I+b relationship, cf. FIG. 1 where as many as 479 tests of K⁺ and ccfHb, including triplicate measurements in each of the 479 tests, were carried out. In brief, the more measurement points, the more accurate the regression model will be, (cf. the R²=0.9907 in FIG. 1). Typically, the total amount of measurements in steps B and C will be at least 3.

It is, however, in some cases possible that the regression or curve fitting model has to be non-linear. For instance, if the analyte itself is bound to the intracellular marker in a state of equilibrium, the gradual release of the intracellular marker need not be accompanied by a linear increase or decrease in the analyte concentration. It is however possible to gauge whether such more complex events are relevant and then derive the best non-linear regression model for that purpose.

In practice, the deliberate lysing of cells in steps B and C can be attained with any practical method known in the art: For instance, a physical treatment of the sample selected from the group consisting of mechanical shearing such as homogenization or bead mill treatment, ultrasound treatment, freeze-thaw treatment, heating, osmotic shock treatment, and cavitation. Alternatively, a chemical treatment is selected from the group consisting of detergent treatment, alkaline treatment, and enzyme treatment. Since chemical treatment by nature will dilute the sample tested, appropriate correction for the dilution may have to be included in the measurement of the concentration of analyte and intracellular maker.

Importantly, lysis of cells can take place after obtaining a sample from a subject, and in addition some of the analytes of interest will after some time begin to equilibrate between the intracellular and extracellular compartment due to declining activity of active transport mechanisms across the plasma membrane in the cells of the sample; the time lapsing before such equilibration takes place depends i.a. on the temperature, where a low temperature can slow down the passive transport across the plasma membrane. So, preferably the sample containing blood cells is obtained from a subject, preferably a human subject, and the duration of the time interval between obtaining the sample from the subject and carrying out steps A-D is sufficiently short so as to prevent that the concentration of the analyte equilibrates to a significant degree between the extracellular phase and the intracellular phase in the sample. This time interval is typically <1 hour, such as <55 minutes, <50 minutes, <45 minutes, <40 minutes, <35 minutes, <30 minutes, <25 minutes, <20 minutes, <15 minutes, <10 minutes, <9 minutes, <8 minutes, <7 minutes, <6 minutes, <5 minutes, <4 minutes, <3 minutes, <2 minutes, <1 minute, and preferably between 5 and 55 seconds, between 5 and 50 seconds, between 5 and 45 seconds, between 10 and 45 seconds, and between 15 and 45 seconds. In brief, the faster the sample is subject to the method of the 1^{st} aspect of the invention, the more accurate the regression model will be for the purpose of providing precise determinations of the impact of cell lysis.

An attractive feature of the first aspect of the invention is that it can be carried out for multiple analytes in parallel; in that case, the impact on the measured concentration of each one of multiple analytes is determined and step D then defines the relationships between changes in the concentration of each of the multiple analytes and the concentration of the intracellular marker. In practice, a sample is treated as described above in the embodiments of the first aspect, but instead of only establishing one regression model, multiple regression models are established, one for each analyte. For example, a sample can be tested for K⁺, Na⁺, Ca²⁺, and ccfHb a number of times, and the measured ccfHb values are then fitted into regression models against the K⁺, Na⁺, and Ca²⁺ values.

Conveniently, the method of the 1^{st} aspect is carried out in a device, which is adapted to measure the concentration of the intracellular marker and the concentration of the analyte or, when relevant, the multitude of analytes. This will facilitate rapid and efficient measurement and read-out of results. However, while not preferred the method can also be carried out in at least 2 separate devices, wherein one of the at least 2 separate devices is adapted to measure the concentration of the intracellular marker, and wherein the remaining of the at least 2 devices is/are adapted to measure the analyte or, where relevant, the multitude of analytes. The latter requires that calculation of the impact of cell lysis is then carried out manually, based on read-outs from the at least 2 separate devices, or that the measurement values are—at some point—all input (manually or via a communication means) into the same device, which carries out the necessary calculation.

Typically, a device for carrying out the method of the 1^{st} aspect comprises
inlet(s) for a cell-free liquid comprising or consisting of extracellular phase material or
inlet(s) for a sample comprising both extracellular phase material and blood cells, and where the device further comprises means for separating cells from the extracellular phase.

If the sample is a blood sample, cells can be separated off the sample input into the measuring device prior to introducing the extracellular phase material into the device, but the device can also include the necessary separation modules: e.g. a filter unit or a centrifuge (which spins down cells and allows that the extracellular material is removed from the spun-down cells) or a coated membrane. Alternatively, cells can be sorted via a porous mirror or via iso-acoustic focusing, such as described in Augustsson, P., Karlsen, J., Su, H W. et al. Iso-acoustic focusing of cells for size-insensitive acousto-mechanical phenotyping. Nat Commun 7, 11556 (2016).

### 2^{nd} Aspect of the Invention

This aspect relates to a method for calibration of a device for determination of extracellular analyte concentration, the method comprising carrying out, with the device, the method according to any one of the first aspect of the invention and any embodiments thereof disclosed herein, storing in a memory module in the device the constant values characterizing the function defined in the 1^{st} aspect of the invention and any embodiments thereof discussed above, so as to allow output based on subsequent measurements of analyte performed with the device to be adjusted on the basis of measured values of the intracellular marker.

Hence, as explained above, the device can be calibrated at an initial stage were the method of the 1^{st} aspect of the invention is carried out and then storing the necessary constant(s) or curve-fitting data from the regression model/curve fitting model in a memory of the device. When subsequent measurements of analyte are carried out and recorded in the device and results of corresponding measurements of the intracellular marker are also recorded in the device, the end user can obtain information about the total amount of analyte in the extracellular fraction of the sample as well as information about the contribution from lysed cells. In turn, this allows for a more accurate clinical evaluation of the data: in most cases, the cytolysis takes place during sampling and the contribution from this artefact can be ruled out already at the measurement stage: a seemingly clinically relevant deviation from a normal value of a measured analyte concentration can be qualified by an indication that the measurement result has been influenced by cell lysis. Likewise, a seemingly normal value of a measured analyte can—after the result has been broken down into a “true” value and a cell lysis contribution value—be flagged as clinically relevant. In other words, the method of the 1^{st} aspect of the invention enables that measurements of analytes provide a more accurate result for the clinician to take into consideration, cf. also the 3^{rd} aspect of the invention.

### 3^{rd} Aspect of the Invention

In line with the above, this aspect provides a method for determination of analyte concentration distribution in the extracellular phase of a sample, the method comprising measuring the total concentration of the analyte in the extracellular phase and measuring the concentration of an intracellular marker in the extracellular phase, and subsequently calculating the contribution from lysis of cells to the total concentration of the analyte by inter- or extrapolation from the measured concentration of the intracellular marker, wherein the mathematical relationship between analyte concentration and concentration of the intracellular marker has been established according to the methods of the 1^{st} and/or 2^{nd} aspects of the invention as well as any embodiments thereof disclosed herein.

In line with the method of the 1^{st} aspect and the embodiments thereof, such a determination of analyte concentration distribution should preferably be carried out to avoid that the analyte is subject to equilibration between the intra- and extracellular compartments—or at the very least that such equilibration does not exceed what would have been the case when carrying out the methods of the 1^{st} and 2^{nd} aspects of the invention and embodiments thereof. So, preferably the sample containing blood cells is obtained from a subject, preferably a human subject, and the duration of the time interval between obtaining the sample from the subject and carrying out the method of the 3^{rd} aspect of the invention is sufficiently short so as to prevent that the concentration of the analyte equilibrates to a significant degree between the extracellular phase and the intracellular phase in the sample, and preferably the duration is about the same as that utilized when determining the mathematical relationship with the method of the 1^{st} or 2^{nd} aspect of the invention. So also here, the time interval is typically <1 hour, such as <55 minutes, <50 minutes, <45 minutes, <40 minutes, <35 minutes, <30 minutes, <25 minutes, <20 minutes, <15 minutes, <10 minutes, <9 minutes, <8 minutes, <7 minutes, <6 minutes, <5 minutes, <4 minutes, <3 minutes, <2 minutes, <1 minute, and preferably between 5 and 55 seconds, between 5 and 50 seconds, between 5 and 45 seconds, between 10 and 45 seconds, and between 15 and 45 seconds. In brief, the faster the sample is subject to the method of the 3^{st} aspect of the invention, the more accurate the determination of the contribution from cytolysis will be and the more accurate the correction of the measurement of the analytes will be.

As mentioned under the 2^{nd} aspect, the determination of the analyte concentration distribution provides the skilled person with a much more accurate set of data for later determination of steps to be taken in the clinical setting.

### 4^{th} Aspect of the Invention

This aspect relates to a device for determination of analyte concentration distribution in the extracellular phase of a sample (such as a measurement sample), wherein the device is configured to measure the total concentration of the analyte in the extracellular phase and measure the concentration of an intracellular marker in the extracellular phase, and subsequently estimating, such as calculating, the contribution from lysis of cells to the total concentration of the analyte by inter- or extrapolation from the measured concentration of the intracellular marker based on a mathematical or functional relationship between analyte concentration and concentration of the intracellular marker, such as established according to the method of any one of the 1^{st} and 2^{nd} aspects.

The device may be advantageous for enabling determining a true extracellular concentration of an analyte in a sample, such as the extracellular concentration of the analyte in a sample disregarding the contribution from lysis of cells to the total concentration of the analyte.

The ‘sample’, such as the measurement sample, may in this context be understood to be a sample for which a true extracellular concentration of an analyte is to be determined.

A ‘device’ is understood to encompass both distributed devices, such as a system comprising a plurality of separate (sub-) devices, and single devices, such as a unit in a single casing.

The device may in embodiments be a unit, such as a unit encompassed in a single casing and optionally with a single sample inlet and/or a common sample inlet for a sample for which the concentration of the intracellular marker and an analyte concentration is measured.

The device may in embodiments be understood to be a distributed device (such as a system), such as two or more physically separated (sub-) devices. Said (sub-) devices may be digitally connected to each other and/or to a processing unit, such as a processing unit arranged for estimating, such as calculating, the contribution from lysis of cells to the total concentration of the analyte by inter- or extrapolation from the measured concentration of the intracellular marker based on a mathematical or functional relationship between analyte concentration and concentration of the intracellular marker.

The device may comprise a (sub-) device, which is adapted to measure the concentration of the intracellular marker and the concentration of the analyte or, when relevant, the multitude of analytes.

The device may comprise at least 2 separate (sub-) devices, wherein one of the at least 2 separate (sub-) devices is adapted to measure the concentration of the intracellular marker, and wherein the remaining of the at least 2 (sub-) devices is/are adapted to measure the analyte or, where relevant, the multitude of analytes.

The device may comprise an:
inlet(s) for a cell-free liquid comprising or consisting of extracellular phase material or
inlet(s) for a sample comprising both extracellular phase material and blood cells, and where the device further comprises means for separating cells from the extracellular phase, such as wherein the means for separating cells from the extracellular phase is selected from a filter (such as a porous mirror (PM)) or a centrifuge or a porous mirror or iso-acoustic focusing.

The device may comprise or be operatively coupled to a memory module storing values characterizing the mathematical or functional relationship, such as so as to allow output based on measurements of analyte performed with the device to be adjusted on the basis of measured values of the intracellular marker based on the mathematical or functional relationship.

According to an embodiment, the device is further arranged for determining the mathematical or functional relationship by determination of the impact from cell lysis on a measured concentration of an analyte present in a blood cell-containing liquid sample, by
I) the device receiving, optionally sequentially, a plurality of blood cell-containing liquid samples, wherein the plurality of blood cell-containing liquid samples differ with respect to each other in fraction of lysed blood cells,
II) the device measuring the concentration of the analyte in the extracellular phase from each sample containing blood cells and measuring the concentration of an intracellular marker in the extracellular phase of each sample,
III) the device defining, such as via external units, such as via cloud computing, the mathematical or functional relationship in a regression model or a curve-fitting model, in the form of a function where the concentration of the analyte is the dependent variable and the concentration of the intracellular marker is the independent variable, the relationship between changes in the analyte concentration and the concentration of the intracellular marker by using the pairs of measurements obtained in step II.

By ‘the device defining’ in step III, is to be understood that the device may encompass distributed systems, such as the device or a part of the device being arranged for defining or having defined the mathematical or functional relationship, e.g., by requesting another part of the device or another unit to carry out defining the mathematical or functional relationship.

According to this (calibration) embodiment, the device is further arranged for receiving a plurality of samples, optionally simultaneously or sequentially, which differ in lysis fraction, such as wherein a blood sample has been aliquoted and each aliquot has been (optionally manually) lysed to a unique degree, or wherein sub-samples are drawn from a sample which is (optionally manually) lysed to an increasingly larger degree between drawing of each sub-sample, and wherein the aliquots or sub-samples are provided to the device simultaneously or sequentially.

The ‘(blood cell-containing liquid) sample’, such as the (blood cell-containing liquid) sample being a calibration sample, may in this context be understood to be a sample for use in determining (calibrating) the mathematical or functional relationship. This (calibration) sample may or may not be identical with the (measurement) sample for which a true extracellular concentration of an analyte is to be determined. The plurality of blood cell-containing liquid (calibration) samples may thus originate from the same (measurement) sample for which the true extracellular analyte of an analyte is to be measured (in which case the mathematical or functional relationship is expectedly most accurate) or be another sample than the (measurement) sample (in which case the relationship can be considered an approximation).

According to an embodiment, the device is further arranged for determining the mathematical or functional relationship by determination of the impact from cell lysis on a measured concentration of an analyte present in a blood cell-containing liquid sample, by
i) the device receiving a blood cell-containing liquid sample,
ii) the device measuring the concentration of the analyte in the extracellular phase from the sample containing blood cells or a part thereof and measuring the concentration of an intracellular marker from the sample containing blood cells or a part thereof,
iii) the device lysing a fraction of the blood cells followed by measuring a concentration of the analyte and a concentration of the intracellular marker in the extracellular phase of the sample containing the lysed cells,
iv) the device optionally repeating step iii one or more times, and
v) the device, such as via external units, such as via cloud computing, defining the mathematical or functional relationship in a regression model or a curve-fitting model, in the form of a function where the concentration of the analyte is the dependent variable and the concentration of the intracellular marker is the independent variable, the relationship between changes in the analyte concentration and the concentration of the intracellular marker by using the pairs of measurements obtained in steps ii-iv.

By ‘the device defining’ in step V, is to be understood that the device may encompass distributed systems, such as the device or a part of the device being arranged for defining or having defined the mathematical or functional relationship, e.g., by requesting another part of the device or another unit to carry out defining the mathematical or functional relationship.

According to this (calibration) embodiment, the device if further arranged for receiving a sample and (optionally automatically) lyse varying fractions of the blood cells and measure corresponding values of analyte and intracellular marker. A possible advantage of this embodiment may be that calibration and/or determination of the mathematical or functional relationship may be done faster, involving less (human) resources and/or more consistently. Lysis may be done by the apparatus, e.g., by applying heat, shear forces, shaking, pressure changes or cavitation or ultrasound, such as wherein the apparatus comprises a heating unit, a narrow or capillary channel, a vortex mixer, a channel with a narrowing portion for inducing a pressure change (such as cavitation) upon fluid passing through the channel, a piston connected to a confined space and/or an ultrasound source.

According to an embodiment, the device comprises an optical measurement setup, such as comprising a porous mirror (PM), arranged for measuring the concentration of an intracellular marker in the extracellular phase, and/or an electroanalytical measurement setup, such as an ion selective electrode sensor, such as an ion selective electrode membrane sensor, for measuring the total concentration of the analyte in the extracellular phase.

### 5^{th} Aspect of the Invention

According to this aspect there is presented a computer program or computer program product comprising instructions to cause the device of the 4^{th} aspect to measure the total concentration of the analyte in the extracellular phase of a sample and to measure the concentration of an intracellular marker in the extracellular phase of a sample, and subsequently to estimate, such as calculate, the contribution from lysis of cells to the total concentration of the analyte by inter- or extrapolation from the measured concentration of the intracellular marker based on a mathematical or functional relationship between analyte concentration and concentration of the intracellular marker.

According to an embodiment, the computer program or computer program product may furthermore be comprising instructions to cause the device of the 4^{th} aspect to
i) measure the concentration of the analyte in the extracellular phase of a sample from each of a plurality of samples containing blood cells and measure the concentration of an intracellular marker in the extracellular phase of each sample (and optionally cause the device to do lysis of the sample so that samples within the plurality of samples differ in fraction of lysed cells with respect to each other), and
ii) define the mathematical or functional relationship in a regression model or a curve-fitting model, in the form of a function where the concentration of the analyte is the dependent variable and the concentration of the intracellular marker is the independent variable, the relationship between changes in the analyte concentration and the concentration of the intracellular marker by using the pairs of measurements obtained in step i.

According to this aspect the invention relates to a computer program or computer program product comprising instructions to cause the device of the 4^{th} aspect to carry out certain steps, such as a computer program product being adapted to enable a processor or a computer system comprised within the device of the 4^{th} aspect, comprising at least one computer having data storage means in connection therewith to control the device of the 4^{th} aspect. This aspect of the invention is particularly, but not exclusively, advantageous in that the present invention may be accomplished by a computer program or a computer program product enabling the device of the 4^{th} aspect to carry out the operations of the device of the when down- or uploaded into the device, such as into a computer system comprised within the device of the 4^{th} aspect. Such a computer program or computer program product may be provided on any kind of computer readable medium, or through a network.

### Considerations Relevant for all Aspects of the Invention

General Measurement Principles, with Special Relevance for K⁺ and Other Ions.

The measuring principles for the electrolyte sensors in an exemplary K⁺ measurement device (such as the ABL90 FLEX analyzer from Radiometer) is based on an electrochemical measuring method.

Potentiometry: The potential of an electrode chain is measured by a voltmeter, and related to the concentration of the sample (the Nernst equation). The potentiometric measuring principle is applied in the pH, pCO2 (pH measurement), K⁺, Na⁺, Ca²⁺, and Cl⁻ sensors.

Strictly speaking, in potentiometry the potential of an electrode chain is related to the activity of a substance not its concentration. The activity of a substance can be considered the effective concentration of a species that takes non-ideality of the medium into account.

Activity and concentration are related by ax=γcx, where ax is the activity of the species x, γ=the activity coefficient of species x under the measurement conditions (for ideal systems γ=1), and cx=the concentration of species x (mol/L). To be exact, activity is related to the molality of species x (the amount of substance of the solute (in mol) divided by the mass of the solvent (in kg)). However, molality is converted to concentration (molarity). The ABL90 FLEX analyzer automatically converts activities into concentrations. The term “concentration” is therefore used in explanations of the measuring principles for each of the sensors.

Potentiometric measurement principle: The pH and electrolyte sensors are measured according to the potentiometric measurement principle, where the potential of an electrode chain recorded at a voltmeter is related to the concentration of a substance via the Nernst equation.

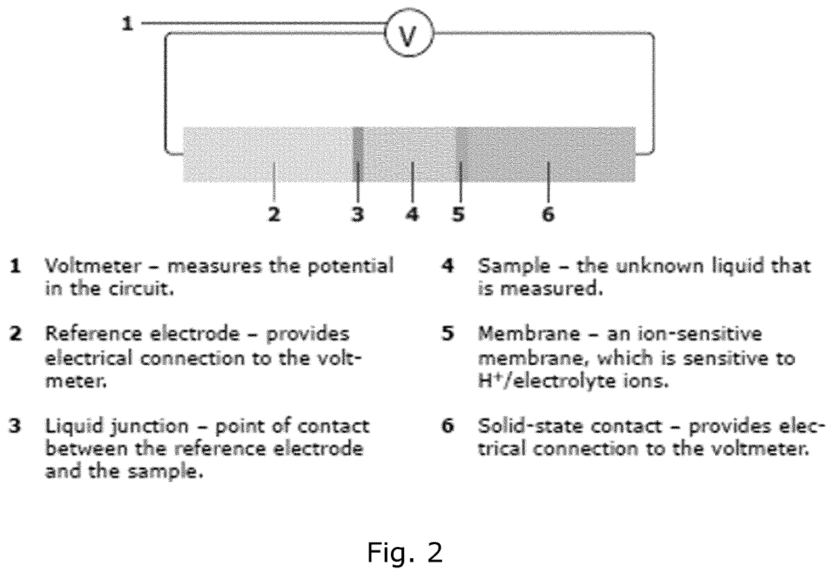

Electrode chain: The electrode chain (or electrical circuit) set up to measure pH/electrolytes is shown in FIG. 2.

As an alternative to the above potentiometric measurement principles, Flame Emission Spectrophotometry can be used (L. J. Kricka, J. Y. Park, in Pathobiology of Human Disease, 2014). Flame emission spectrophotometry is based on the characteristic emission of light by atoms of many metallic elements when given sufficient energy, such as that supplied by a hot flame. The wavelength to be used for the measurement of an element depends on the selection of a line of sufficient intensity to provide adequate sensitivity and freedom from other interfering lines at or near the selected wavelength. For example, lithium produces a red, sodium a yellow, potassium a violet, rubidium a red, and magnesium a blue color in a flame. These colors are characteristic of the metal atoms that are present as cations in solution. Under constant and controlled conditions, the light intensity of the characteristic wavelength produced by each of the atoms is directly proportional to the number of atoms that are emitting energy, which in turn is directly proportional to the concentration of the substance of interest in the sample. Although this technique once was used for the analysis of sodium, potassium, and lithium in body fluids, it has been replaced largely by electrochemical techniques.

Measurement Principles for ccfHb:

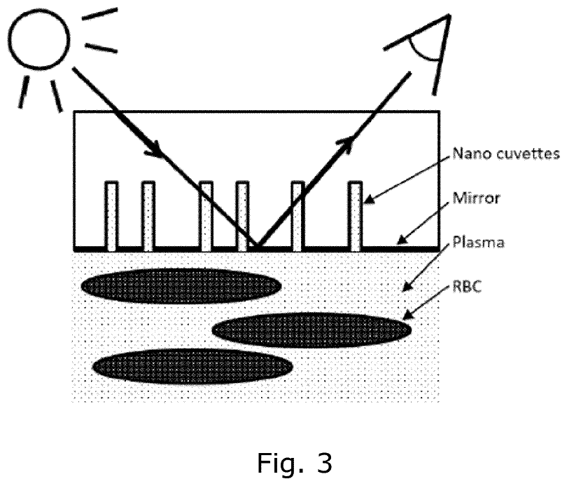

Reference is made to FIG. 3, which shows a diagram of the porous mirror (PM) sensor construction. In brief the porous mirror (PM) is a proprietary technology to provide an optical absorbance measurement of the plasma phase of a whole blood (WB) sample. It is used to determine the concentration of cell free haemoglobin (ccfHb). It functions by allowing ccfHb to diffuse into a porous PETP membrane, with a pore size smaller than the red blood cells (RBCs). RBCs are thus excluded from entering into the pores. The pores are dead-end inside the membrane, so each of the 1.2 million pores per mm² constitutes a nano-cuvette (ø=400 nm, length=25 μm) in close proximity to the sample. ccfHb and other plasma constituents are transported to and from the nano-cuvettes by diffusion. The front side (facing the sample) of the porous membrane is coated with a noble metal (Pd, thickness=100 nm), but still open at the pore ends facing the sample. The metal layer at the front of the membrane allows for light reflection (the mirror in PM) enabling a transmission-like measurement of the haemoglobin (Hb) inside the pores from the backside of the membrane. Conveniently, optical interferences from Hb in the RBCs in the sample and/or other particulate matter are suppressed to a negligible level by the same optically shielding metal layer. The porous mirror device may be a porous mirror for detection of an analyte in a fluid by optical probing, comprising
a translucent slab with a front side, and a backside facing away from the front side, wherein the front side is adapted for being contacted with a fluid, and
a reflective layer at the front side of the translucent slab, the reflective layer being adapted to reflect light reaching the reflective layer from the backside of the translucent slab,
   wherein the translucent slab comprises pores, wherein the pores are dead end pores, extending from respective openings at the front side into the translucent slab, through the reflective layer,
   wherein a cross-sectional dimension of the openings of the pores is dimensioned so as to prevent larger particles or debris, if any included the fluid, from entering the pores, while allowing the analyte in the fluid to enter the pores via diffusion. The porous mirror (PM) technology is described in WO 2017/085162 A1, which is hereby incorporated in entirety, such as particularly described in claim 1 of WO 2017/085162 A1 and furthermore in FIG. 1 and the accompanying text on pages 24-25.

Alternatively, one may utilize the standard or reference method for determination of the haemoglobin concentration, which is the HiCN method: blood is mixed with a solution of potassium cyanide, potassium ferricyanide, and Drabkin's solution, the erythrocytes are lysed by producing an evenly disturbed haemoglobin solution. Potassium ferricyanide transforms haemoglobin into methaemoglobin (i.e. a haemoglobin derivative), and methaemoglobin combines with potassium cyanide to produce haemiglobincyanide (cyanmethaemoglobin). When the reaction is entire, the absorbance of the solution is deliberate in a spectrophotometer at 540 nanometers. haemoglobincyanide has a wide absorbance peak at this wavelength. The absorbance is compared with that of the standard haemiglobincyanide solution by using a formula to obtain the amount of haemoglobin.

This method is optional for the estimation of haemoglobin and this method is recommended by the International Committee for Standardization in haematology. This is because in this method all type of haemoglobin is transformed to cyanmethaemoglobin (except sulfhaemoglobin), and a firm and trustworthy standard is available.

### Exemplary Device

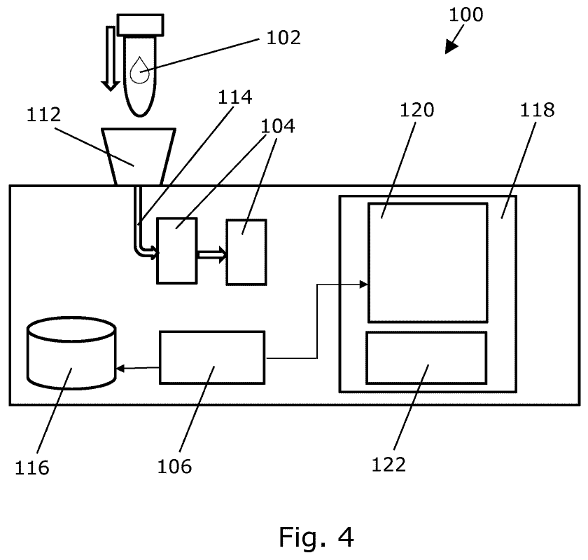

FIG. 4 is a schematic illustration of an exemplary device 100 for determination of analyte concentration distribution in the extracellular phase of a (liquid) sample (such as a measurement sample) 102 comprising the analyte and an intracellular marker, being cell-free haemoglobin, wherein the device is configured to measure the total concentration of the analyte in the extracellular phase and measure the concentration of an intracellular marker in the extracellular phase, and subsequently estimating, such as calculating, the contribution from lysis of cells to the total concentration of the analyte by inter- or extrapolation from the measured concentration of the intracellular marker based on a mathematical or functional relationship between analyte concentration and concentration of the intracellular marker, such as established according to the method of any one of the 1^{st} and 2^{nd} aspects, said device comprising:
one or more sensors (or sub-devices) 104 for measuring:
   the analyte concentration in the liquid sample, and
   a cell-free haemoglobin concentration in the liquid sample,
a data processing device 106 comprising a processor configured to measure the total concentration of the analyte in the extracellular phase and to measure the concentration of an intracellular marker in the extracellular phase, and subsequently to estimate, such as calculate, the contribution from lysis of cells to the total concentration of the analyte by inter- or extrapolation from the measured concentration of the intracellular marker based on a mathematical or functional relationship between analyte concentration and concentration of the intracellular marker.

In the depicted embodiment, the one or more sensors 104 comprise two sensors, one for measuring each of the analyte concentration in the liquid sample, and the cell-free haemoglobin concentration in the liquid sample. The schematic illustration in FIG. 4 furthermore shows a liquid sample inlet 112, a microfluidic system 114, a memory module 116, a user interface 118, wherein the user interface comprises an output unit 120 arranged for visually outputting information relating to any one or more or all of the total concentration of the analyte, the concentration of the intracellular marker, the contribution from lysis of cells to the total concentration of the analyte, the true concentration of the intracellular marker, and the mathematical or functional relationship. The thin-line arrows indicate flow of information, such as the analyte concentration in the liquid sample and the cell-free haemoglobin concentration in the liquid sample flowing from the one or more sensors 104 to the data processing device 106, the output signal flowing from the data processing device 106 to the user interface 118 (and more particularly the output unit 120).

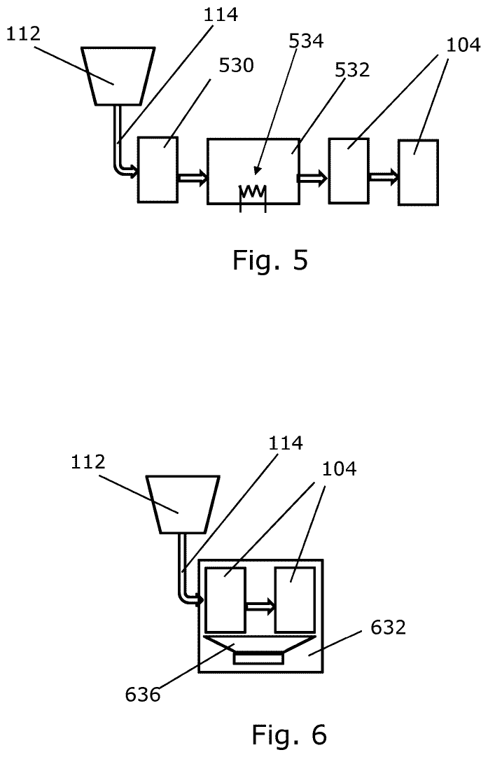

FIG. 5 shows part of an embodiment of an apparatus similar to the device of FIG. 4 comprising the same features and where like reference signs denote like features, but where the apparatus depicted in FIG. 5 furthermore comprises a lysing unit 532 arranged for lysing a fraction of the blood cells (in the sample or a portion thereof), such as a chamber with a lysing element. The apparatus in FIG. 5 furthermore comprises a sample reservoir 530. In the specific embodiment depicted in FIG. 5, the lysing unit comprises a lysing element in the form of a heating device 534 arranged for heating the sample in the lysing unit, but the heating device could be replaced with, e.g., an ultrasound device, one or more capillaries, a vortex mixer or a piston. During use, a sample may be kept in the sample reservoir and portions of the sample may be moved or drawn sequentially through the lysing unit, where the portions may be lysed to varying degree, e.g., a first portion is lysed to zero or a minimum degree by the lysing unit 532 being inactive during passage of said portion through the lysing unit, whereas subsequent portions are lysed to still larger degree due to the lysing unit being active to a still larger degree (e.g., via increased temperature or prolonged time of the sample in the lysing unit) during passage of said portions through the lysing unit. The passage through the lysing unit is followed by measuring a concentration of the analyte and a concentration of the intracellular marker in the extracellular phase of the sample containing the lysed cells.

In an alternative embodiment (not shown), the separate sample reservoir 530 can be dispensed with, e.g., due to the lysing unit overtaking the function of the separate sample reservoir, such as the sample being kept in the lysing unit and lysed therein to a still larger degree (over time), while portions of the sample are drawn sequentially form the lysing unit, thereby achieving spatially and temporally separated portions of a sample differing in the fraction of lysed cells.

FIG. 6 shows another embodiment of an apparatus similar to the device of FIG. 4 comprising the same features and where like reference signs denote like features, but where the apparatus depicted in FIG. 6 furthermore comprises a lysing unit 632 arranged for lysing a fraction of the blood cells (in the sample or a portion thereof). In FIG. 6, the chamber of the lysing unit may perform the function of the separate sample reservoir of FIG. 5 and comprise elements for performing both lysing and measuring, such as a lysis element and sensors 104). In the apparatus in FIG. 6 the lysing unit is arranged for lysing a fraction of the blood cells while being in the chamber(s) of sensors 104 (which may also be the chamber of the lysing unit). In the specific embodiment depicted in FIG. 6, the lysing unit comprises a lysing element in the form of an ultrasound source 636 arranged for exposing the sample to ultrasound, but the ultrasound source could be replaced with, e.g., a heater, capillaries, a vortex mixer or a piston. During use, a sample may be kept in the chamber(s) of the sensors, where the sample may be lysed to varying degree between sequential measurements, e.g., a first measurement is done before while the sample is lysed to zero or a minimum degree by the lysing unit 632, e.g. due to the lysing unit being inactive prior and during said first measurement, whereas before or during subsequent measurements the sample is lysed to a still larger degree, e.g., due to the lysing unit being active and the sample consequently being lysed to an increasing degree as time passes between (and optionally during) measurements. Measurements include measuring a concentration of the analyte and a concentration of the intracellular marker in the extracellular phase of the sample containing the lysed cells.

1. A method for determination of an impact from cell lysis on a measured concentration of an analyte present in a blood cell-containing liquid sample, the method comprising:
A) measuring the concentration of the analyte in an extracellular phase from a sample containing blood cells and measuring an concentration of an intracellular marker in the extracellular phase of the sample,
B) subsequently lysing a fraction of the blood cells followed by measuring a concentration of the analyte and a concentration of the intracellular marker in the extracellular phase of the sample containing the lysed cells,
and
C) estimating a mathematical relationship between changes in the analyte concentration and the concentration of the intracellular marker at least on the basis of the pairs of measurements obtained in steps A-B,
whereby the impact by cell lysis on measured concentrations of the analyte in samples is determined by calculating a part of the analyte concentration derived from cell lysis from a measured concentration of the intracellular marker.

2. The method according to claim 1, wherein the analyte is an ion or electrolyte.

3. The method according to claim 2, wherein the analyte is or comprises an ion selected from K⁺, Na⁺, Ca2+, Mg2+, and Cl.

4. The method according to claim 1, wherein the analyte is a non-electrolyte selected from the group consisting of lactate dehydrogenase (LD), aspartate aminotransferase (AST), bilirubin, Albumin, alkaline phosphatase (ALP), amylase, HDL-cholesterol, creatine kinase (CK), glucose, total serum or plasma protein, triglycerides, unsaturated iron binding capacity (UIBC) and uric acid.

5. The method according to claim 1, wherein the sample in step A is a blood sample.

6. The method according to claim 1, wherein the cell lysis is haemolysis.

7. The method according to claim 1, wherein the intracellular marker is haemoglobin or a haemoglobin derivative.

8. The method according to claim 7, wherein the intracellular marker is cell-free haemoglobin.

9. The method according to claim 1, wherein lysing of cells in step B is a physical treatment selected from the group consisting of mechanical shearing, ultrasound treatment, freeze-thaw treatment, heating, osmotic shock treatment, cavitation, or a chemical treatment selected from the group consisting of detergent treatment, alkaline treatment, and enzyme treatment.

10. The method according to claim 1, wherein the sample containing blood cells is obtained from a subject, and wherein the duration of the time interval between obtaining the sample from the subject and carrying out steps A-C is sufficiently short so as to prevent the concentration of the analyte equilibrating between the extracellular phase and the intracellular phase in the sample.

11. The method according to claim 1, wherein an impact on the measured concentration of each one of multiple analytes is determined and where step C defines the mathematical relationships between changes in the concentration of each of the multiple analytes and the concentration of the intracellular marker.

12. The method according to claim 1, wherein step B is carried out one or more times on the same sample from one individual.

13. The method according to claim 1, wherein a total impact of cell lysis on measured concentrations is determined by calculating a part of the analyte concentration is derived from total cell lysis.

14. A method of calibration of a device for determination of extracellular analyte concentration, the method comprising:
A) carrying out, with the device, the method according to claim 1, and
B) storing in a memory module in the device a mathematical expression of the relationship between changes in the analyte concentration and the concentration of the intracellular marker defined in claim 1,
so as to allow output based on subsequent measurements of analyte performed with the device to be adjusted on the basis of measured values of the intracellular marker.

15. The method according to claim 14, wherein the device is calibrated at an initial stage according to the method of claim 1, and wherein constant(s) or curve-fitting data from a regression model/curve fitting model is stored in a memory of the device.

16. A method for determination of analyte concentration distribution in an extracellular phase of a sample, the method comprising measuring the total concentration of the analyte in the extracellular phase and measuring the concentration of an intracellular marker in the extracellular phase, and subsequently calculating a contribution from lysis of cells to the total concentration of the analyte or correcting the value of the measured concentration of the analyte by inter- or extrapolation from the measured concentration of the intracellular marker, wherein a mathematical or functional relationship between analyte concentration and concentration of the intracellular marker has been established according to the method of claim 1.

17. A device for determination of analyte concentration distribution in an extracellular phase of a sample, wherein the device is configured to measure a total concentration of the analyte in the extracellular phase and measure a concentration of an intracellular marker in the extracellular phase, and subsequently estimate a contribution from lysis of cells to the total concentration of the analyte by inter- or extrapolation from the measured concentration of the intracellular marker based on a mathematical or functional relationship between analyte concentration and concentration of the intracellular marker.

18. The device according to claim 17, further arranged for determining the mathematical or functional relationship by determination of the impact from cell lysis on a measured concentration of an analyte present in a blood cell-containing liquid sample, by:
i) the device receiving a blood cell-containing liquid sample,
ii) the device measuring the concentration of the analyte in the extracellular phase from the sample containing blood cells or a part thereof and measuring the concentration of an intracellular marker from the sample containing blood cells or a part thereof,
iii) the device lysing a fraction of the blood cells followed by measuring a concentration of the analyte and a concentration of the intracellular marker in the extracellular phase of the sample containing the lysed cells,
and
iv) the device defining the mathematical or functional relationship in a regression model or a curve-fitting model, in the form of a function where the concentration of the analyte is the dependent variable and the concentration of the intracellular marker is the independent variable, the relationship between changes in the analyte concentration and the concentration of the intracellular marker by using the pairs of measurements obtained in steps ii-iii.

19. The device according to claim 17, wherein the device comprises one or both of:
i) an optical measurement setup arranged for measuring the concentration of an intracellular marker in the extracellular phase, or
ii) an electroanalytical measurement setup, for measuring the total concentration of the analyte in the extracellular phase.

20. A computer program or computer program product comprising instructions to cause the device of claim 17 to measure the total concentration of the analyte in the extracellular phase of a sample and to measure the concentration of an intracellular marker in the extracellular phase of a sample, and subsequently to estimate, the contribution from lysis of cells to the total concentration of the analyte by inter- or extrapolation from the measured concentration of the intracellular marker based on a mathematical or functional relationship between analyte concentration and concentration of the intracellular marker.
